(19) United States
(12) Patent Application Publication
(10) Pub. No.: US 2016/0310524 A1
(43) Pub. Date: Oct. 27, 2016

(54) COMPOSITIONS AND METHODS FOR TREATING DYSREGULATED SYSTEMS

(71) Applicant: Ralph Ankenman, Sherman Oaks, CA (US)

(72) Inventor: Ralph Ankenman, Sherman Oaks, CA (US)

(21) Appl. No.: 15/104,238

(22) PCT Filed: Dec. 13, 2014
(86) PCT No.: PCT/US14/70196

§ 371 Date: Jun. 13, 2016

Related U.S. Application Data
(60) Provisional application No. 61/915,935, filed on Dec. 13, 2013.

(51) Int. Cl. A61K 33/00; A61K 31/4515; A61K 31/13; A61K 31/198; A61K 31/517; A61K 31/519; A61K 31/496; A61K 31/5377; A61K 31/4168; A61K 31/137; A61K 45/06; A61K 31/138

(52) U.S. Cl. CPC A61K 33/00; A61K 45/06; A61K 31/4515; A61K 31/13; A61K 31/198; A61K 31/138; A61K 31/519; A61K 31/496; A61K 31/5377; A61K 31/4168; A61K 31/137; A61K 31/517

(57) ABSTRACT

A method of treating a subject having a disorder or indication includes administering to the subject a minimal dose of a medicament necessary to alleviate the disorder based on action of the medicament. Some embodiments of the present invention include a method of treating a subject having a disorder or indication, including administering to the subject a minimal dose of a medicament necessary to alleviate the disorder based on action of the medicament.

### CROSS-REFERENCE TO RELATED APPLICATION(S)

The present application claims priority to and the benefit of U.S. Provisional Application Ser. No. 61/915,935 filed on Dec. 13, 2013, the entire contents of which are incorporated herein by reference.

### BACKGROUND

Common linear use of medications is designed to saturate the blood at a particular level of the medication. The recommended dosage of prescription drug medications is often set by a pre-determined serum level. By following this linear regime, practitioners commonly write drug prescriptions with an intentional mg/kg concentration in order to produce a specific blood serum level in a patient. Furthermore, the action of the prescribed medication for the primary indication may often induce a dysregulation in the patient, and this dysregulation may further burden the patient in addition to, or in place of the primary indication.

Additionally, diseases and disorders characterized by a receptor upregulation or downregulation, are often treated to counter this effect. However, a single drug acting to upregulate or downregulate a response can often lead to a dysregulation or overcorrection of the patient's system. In some cases, the dysregulation caused by a particular medication is commonly irreversible—i.e. tardive dyskinesia.

Thus, there is a need for treatment regimes that properly treat these diseases, disorders, and conditions so as to enable normal homeostasis to be achieved, allowing for the eventual discontinuation or reduction of medication. The invention is directed to these and other important needs.

### SUMMARY

Some embodiments of the present invention include a method of treating a subject having a disorder or indication, including administering to the subject a minimal dose of a medicament necessary to alleviate the disorder based on action of the medicament.

Some embodiments of the present invention include a composition for treating herpes simplex virus, the composition including an effective dose of lithium carbonate that does not induce effects selected from the group consisting of tremor of the fingers, polyuria, sodium depletion, and central nervous system dysfunction.

Some embodiments of the present invention include a method of treating herpes simplex virus in a subject infected with herpes simplex virus, the method including administering lithium carbonate to the subject.

Some embodiments of the present invention include a composition for treating an imbalance of dopamine in a subject having an imbalance of dopamine, without inducing post-synaptic sensitivity in the subject, including an effective dose of haloperidol, and an effective dose of amantadine.

Some embodiments of the present invention include a composition for treating an imbalance of dopamine in a subject having an imbalance of dopamine, without inducing post-synaptic sensitivity in the subject, including an effective does of alpha-methyl-para-tyrosine, metyrosine (AMPT).

Some embodiments of the present invention include a composition for treating an imbalance of dopamine in a subject having an imbalance of dopamine, without inducing a histaminergic response, including an effective does of amantadine.

Some embodiments of the present invention include a composition for treating a subject having obsessive compulsive disorder (OCD) and/or autism spectrum social disorder without blocking dopamine, including an effective dose of fluoxetine, and an effective dose of risperidone or aripiprazole.

Some embodiments of the present invention include a composition for treating a subject with schizophrenic acute psychosis and/or autism spectrum social disorder, without inducing psychosis, including an effective dose of mecamylamine.

Some embodiments of the present invention include a composition for treating a subject with dissociation disorder, without inducing aggression or disinhibition, including an effective dose of memantine.

Some embodiments of the present invention include a composition for treating a subject with hypertension without inducing fluid retention or fatigue in the subject, including an effective dose of propranolol or timolol.

Some embodiments of the present invention include composition for treating a subject having alpha adrenergic fatigue or postural hypotension, without inducing hypotension in the subject, including an effective dose of clonidine or an effective dose of venlafaxine.

Some embodiments of the present invention include a composition for treating reflex sympathetic dystrophy or chronic pain syndrome, including an effective dose of doxazosin, and an effective dose of propranolol.

Some embodiments of the present invention include a composition for treating or reducing incidence of a migraine in a subject without inducing over dilation of blood vessels in the subject, include an effective dose of propranolol; and an effective dose of doxazosin.

Some embodiments of the present invention include a method of treating a subject having a disorder or indication, including administering to the subject a minimal dose of a medicament necessary to alleviate the disorder based on action of the medicament.

Some embodiments of the present invention include a method of treating a subject having a disease, disorder, and/or condition including co-administering to said subject two or more agents, wherein the agents have opposing mechanisms of action.

### BRIEF DESCRIPTION OF THE DRAWINGS

FIG. 1 is a representation of normal homeostasis in a system of a subject. The solid horizontal line represents a “set-point” or average of the system's steady state. The sloped line increases and decrease in the system's functionality. The change in the systems functionality is represented by “delta.”

FIG. 2 is a representation of a diseased state. The solid horizontal line represents the “set-point” of normal homeostasis. The peaks and valleys represent increases (for example, hypersensitivity) and decreases (for example, hyposensitivity) in the system's functionality. The change in the systems functionality is represented by “delta.”

FIG. 3 is a representation of a diseased state wherein the subject is administered a composition that treats and/or prevents, for example, hypersensitivity. The solid horizontal line represents the “set-point” of normal homeostasis. The peaks and valleys represent increases (for example, hypersensitivity) and decreases (for example, hyposensitivity) in the system's functionality. The change in the systems functionality is represented by “delta.” Although the delta is lowered, hyposensitivity is not addressed.

FIG. 4 is a representation of a diseased state wherein the subject is administered a composition designed to treat and/or prevent, for example, both hypersensitivity and hyposensitivity. The solid horizontal line represents the “set-point” of normal homeostasis. The peaks and valleys represent increases (for example, hypersensitivity) and decreases (for example, hyposensitivity) in the system's functionality. The change in the systems functionality is represented by “delta.” Co-administration of agents having opposing affects reduces the delta and brings the subject's system at or near a set-point.

### DETAILED DESCRIPTION

Aspects of embodiments of the present invention are directed to a tempered regime of medicaments for treating indications and/or diseases. With the implementation of a tempered regime of medicaments, the patient is able to evade dysregulation. As used herein, “indication” refers to a disease or an observed symptom of a patient or subject. In some embodiments, the indication includes at least one symptom that is to be alleviated. In some embodiments, a lower dose of a drug may be administered for treatment of an indication or disease. In some embodiments, the dose of the drug is lower than the lowest used dose of a drug for the same indication or for a different indication. In some embodiments, a drug is administered at a lower dose in combination with a complementary (or opposing action) drug in order to regulate the patient's response and inhibit or decrease dysregulation in the patient.

As used herein, “subject” includes, animals and humans. As used herein, the term “subject” refers to any animal, in particular mammals. Thus, the methods of the invention are applicable to human and nonhuman animals, although it is most preferably used with humans. Thus, in some aspects, the methods are used to treat humans. In other aspects, the methods are used to treat nonhuman animals. The term “subject” does not denote a particular age or sex. Thus, adult, adolescent, child, and newborn subjects, as well as fetuses, whether male or female, are included in the term “subject.”

As used herein, “patient” refers to a subject afflicted with a disease or disorder. The term “patient” includes human and animals.

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The present invention provides methods of treating a subject having a disease, disorder, and/or condition comprising co-administering to said subject two or more agents, wherein the agents have opposing mechanisms of action.

As used herein, the term “treat” refers to the reduction in severity and/or frequency of symptoms, elimination, prevention, and/or amelioration of symptoms and/or the underlying cause, and improvement or remediation of damage. In some aspects of the invention, “treat” refers to reducing the “delta” so as to change and re-set the set-point of the system being treated. “Treat,” “treating,” and “treatment” are used interchangeably herein.

As used herein, the term “co-administrating” refers to the administration of two or more agents together. In some aspects of the invention, the two or more agents are administered simultaneously, or at the same time. In other aspects of the invention, the two or more agents are administered at different times, but are present in the subjects body/system for an overlapping period of time.

As used herein, “opposing mechanisms of action” refers to agents that function in opposite or contradictory ways. For example, in some aspects of the invention, the agents comprising at least one agonist and at least one antagonist, wherein the agonist and antagonist regulate the same target molecule. The target molecule can comprise cellular receptors, ion channels, or both. In other aspects of the invention, at least one agent treats hypersensitivity and at least one agent that treats hyposensitivity. Thus, the present invention includes any combination of drugs, medications, agents, supplements, etc. that are designed to function in opposing manners.

As used herein, the term “hypersensitivity” refers to a greater than normal ability to respond to stimuli. As used herein, the term “hyposensitive” refers to a less than normal ability to respond to stimuli.

Also disclosed herein are methods of treating a subject having a disease, disorder, and/or condition comprising administering to said subject medications according to the diurnal cycle. As used herein, the term “diurnal cycle” refers to any biological process that displays oscillation during a 24 hour period. “Diurnal cycle” and “circadian rhythm” are used interchangeably herein. Thus, in some aspects of the invention, a subject is given one type of agent during the day when the subject is active, and another type of agent during the night when the subject is inactive, such that the agents function in opposing manners.

Delta Dysregulation Disorder

Nature has provided homeostasis as the normal state for many systems in the human body. Delta Dysregulation is a disorder represented by the lack of homeostasis. As a result of this underlying disorder, many symptoms of dysregulated systems appear refractory to treatment.

In the example of hypertension, the most common treatment is a class of medications known as vasodilators. By dilating blood vessels, the volume of the vascular system is increased while the volume of blood does not increase, thus reducing the pressure inside the vascular system. Until now, this one sided treatment of the binary delta of blood pressure has not permitted the patient to reduce or stop taking medication, and blood pressure is primarily regulated when blood vessels are dilated. By applying a treatment protocol using more than one medication (“the Delta Treatment Protocol”) homeostasis can be reestablished by applying vasodilatation and vasoconstriction simultaneously or diurnally, and the vascular system can be regulated more consistently. This would produce a more steady state by treating both sides of the blood pressure delta.

By way of example, migraine headaches are considered a result of vascular spasm in the brain that in turn triggers a neurologic pain response. This vasospasm is most successfully treated with a type of vasodilator specific to migraine in the class of drugs called Triptans. In migraine there is also a delta, where a spasm first dilates, then constricts, the vascular system of the brain in a limited location, producing intense neurologic pain. Treating this type of spasm with only a vasoconstrictor does not address the other side of the delta and therefore invites the headaches to return. By treating both sides of the delta, the possibility of stopping the spasm and the resulting neurologic response entirely is greatly enhanced.

Rational Poly-Pharmacy applies the principles of the Delta Theory to the treatment of disorders, applying lower doses of medication used for related maladies. The inventor has observed that there is not always a direct correlation between the blood serum level of an administered medication once absorbed, and the effectiveness of that particular medication. This phenomenon has led to the formulation of the “Trojan Horse Theory” of medical treatment. If use of medication demonstrates the absence of direct correlation in blood serum levels of a particular drug to its effectiveness in a specific disorder, then it may be concluded that such a result is non-linear.

The “Trojan Horse Theory” describes the efficacy of medications used in low doses. This approach includes overwhelming a molecular target with a lower dose of a medication and/or a combination of lower dose medications, produce significant results in medical treatment using lower dosages.

Re-Regulating Systems to Reset a Dysregulated Set-Point

Resetting a dysregulated set point is accomplished with a strategy of “meeting in the middle.” This requires treating both sides of a dysregulated system. Thus, hypertension can be re-regulated with multiple medications that would never be prescribed together, in order to stop the vascular system from breaking through. Only with this strategic process can true homeostasis return. Once that is achieved, the further goal of removing medication can be accomplished with a strategic process that also applies Delta Theory to retaining homeostasis during the process of titration.

Finding the set-point of a particular disorder is the first step in this process. This is accomplished with application of medication to achieve successful masking of symptoms. Once symptoms are masked and the level of medication achieves the absence of breakthrough, then the re-regulation can begin. This is accomplished by adding the other side of a disorder's delta into treatment. This is how long-term breakthrough is prevented. Once stability has been accomplished with this regimen, it can be stated that homeostasis has been achieved. At this point, using low dose Rational Polypharmacy, the strategic process of titration can begin. In many cases medication can eventually be removed permanently, in some cases where medication must remain in use, the homeostasis becomes permanent.

Regardless of the disorder, the strategic process remains the same or very similar—get the dysregulated system to stabilize around a set point that is achieved by meeting in the middle. This is where Delta Theory distinguishes its process.

An example can be drawn with certain neuropsychiatric disorders such as movement or tic disorders, and applying to post-medication withdrawal dysregulation that may cause disorders such as tardive dyskinesia, as well. In such cases it is understood that the neurotransmitter dopamine plays a major role in producing symptoms, and these symptoms arise from the dysregulation of this neurotransmitter. In normal human homeostasis of dopamine levels, the body produces enough dopamine to keep a steady supply. In dysregulated cases, symptoms appear that reflect changes in the normal balance of dopamine in the brain.

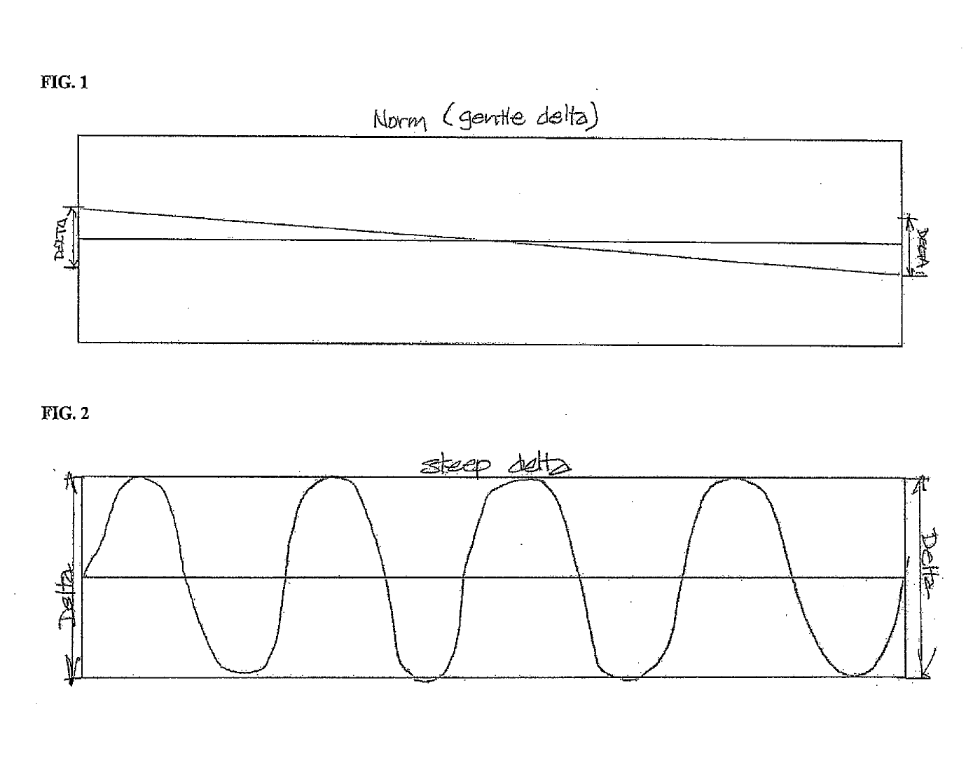

In normal human homeostasis, the level of dopamine produced and made available in the brain remains relatively steady and in balance. It changes little over time and the brain uses what it needs. Thus it can be said that in normal human homeostasis a “set-point” is average for this steady state. It can also be said that the change in dopamine level around this set-point varies little over periods of time, frequently measured in days or weeks. We can describe the variations in level with the mathematical concept of the term “delta”—the difference of the level over time, producing a slope. (FIG. 1)

In dysregulated dopamine patients symptomatic of dopamine related neuropsychiatric disorders, the level of available dopamine and the sensitivity of dopamine receptors changes far more frequently, possibly a number of times in a 24 hour period. (FIG. 2) One result of this swing in dopamine level is the dramatic change in dopamine receptor sensitivity. The existing treatments for the symptoms of this previously unidentified disorder have been mostly one sided, addressing hypersensitivity of the dopamine receptors by setting up a dopamine blockade—a flood-gate to reduce the dopamine being transmitted through the synapse and thus reduce the sensitivity of the dopamine receptors. These neuroleptic medications are in the class of dopamine antagonists.

In the example of Delta Dysregulation Disorder in dopamine dyregulated patients, there is also a hyposensitivity of the receptors. Thus, the change in sensitivity represents a significantly increased variation over a shorter period of time. This can be represented as the delta of dopamine receptor sensitivity, and in the dysregulated patient it is considered “steep”. In such cases, the set-point of dopamine receptor sensitivity has been altered, and this can happen for reasons which include the sudden withdrawal of dopaminergic medications.

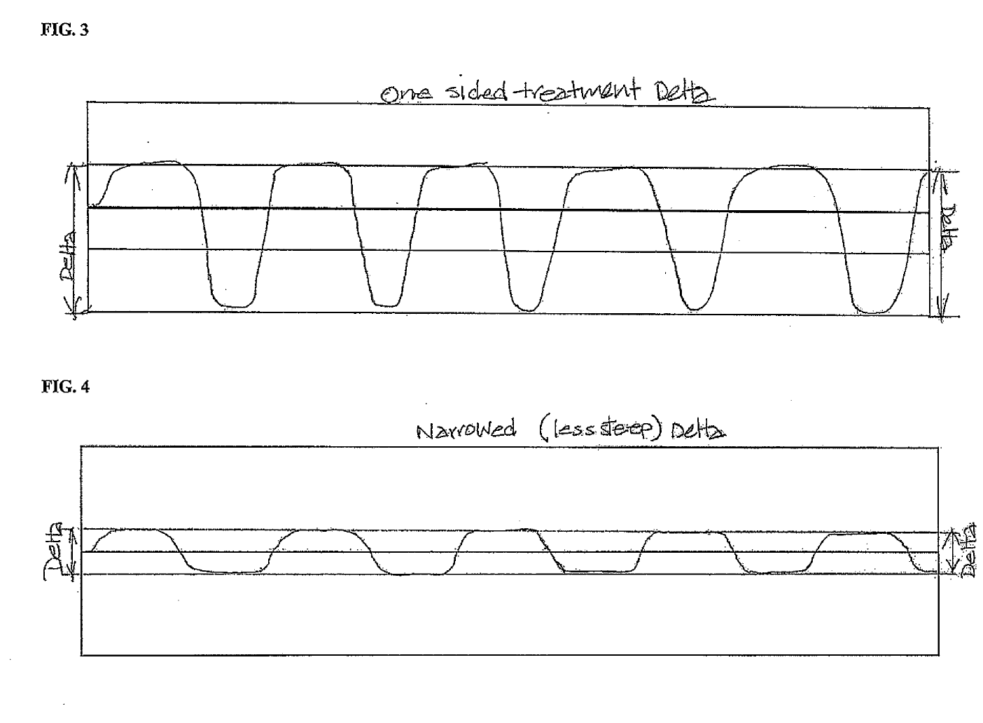

The difference between normal homeostasis and the dysregulated state can be viewed as the difference between a gentle delta slope (in the normal patient) and a steep Delta slope (in the dysregulated patient). In order to successfully treat patients with a Delta Dysregulation Disorder, the steep delta must be addressed from both sides. Common practice in treating symptomatic patients is limited to only one side, the hypersensitive side. (FIG. 3) In early cases where both sides have been treated, the goal was to simply abate symptoms. This treatment was ineffective because the there was no awareness of the existence of the delta causing the underlying problem, therefore they could not achieve homeostasis.

Treatment

In studying the dysregulation of dopamine a process was identified to determine the appropriate treatment to achieve homeostasis that includes pushing the antagonist to the point where side effects such as extra pyramidal symptoms (EPS) appear. In order to find the dysregulated set-point, an excess of medication is given to determine the high point of the delta. It is then reduced quickly to the point where the side effect is not present but the symptom masking remains intact. This is one possible technique to establish the outer limit of set-point dysregulation as defined in Delta Theory.

The treatment of Delta Dysregulation Disorder involves addressing the hyper and hypo-sensitivities of receptors, (e.g., dopamine receptors) simultaneously.

With both sides of the steep delta addressed, it becomes possible to change and re-set the set point of dopamine enabling some patients to eventually no longer require medication, by permitting the slow titration off of some, if not all, pharmaceutical products with the original disorder in complete remission. (FIG. 4)

The application of this theory extends into numerous disorders including, but not limited to, treatment of hypertension, migraine and diabetes. Although these diseases are treated with medications that may or may not apply to other disorders, the principle remains the same: by narrowing a steep delta, the dysregulated differential is reduced, the slope is managed, homeostasis returns by meeting in the middle, and symptoms resolve.

In neuropsychiatry it is possible to improve the efficacy of a dopamine blockade placed with a neuroleptic medication such as haloperidol by using very minor suppression of dopamine production with a low dose of the catecholamine suppressant alpha-methyl-para-tyrosine (AMPT). By making the neuron believe it is getting less dopamine, the neuron stops attempting to breach the dopamine blockade and accessing more, thereby helping the blockade medication remain effective without having to raise the dosage further. This preserves the beneficial results of neuroleptic treatment and adjustment of dysregulation can follow. For example, a very small adjustment to dopamine production—using AMPT as a “Trojan Horse”—tricks the brain into allowing the principal medication to continuously perform its blockade function.

Utilizing the Delta Dysregulation methodology as described herein, the following low dose compositions and methods, including combination therapies, were identified for the following indications and disorders.

Herpes Simplex Virus

One conventional treatment for herpes simplex virus 1 or 2, is to provide antivirals during an outbreak, or as a prophylactic. These medications are given once the virus has already begun to shed. In this way, the antiviral affects the cell in a non-specific way, and stops replication but does not eliminate the disease. Using the Delta Theory, the virus can be trained to cycle predictably by giving lithium in low dose within 36 hours after the outbreak begins, for at least 3 days, to arrest viral shedding. In some embodiments, the antiviral agent may be introduced to complete eradication of the virus. In some embodiments, 30 days after treatment begins, the lithium treatment may be repeated until the set point is re-set.

In some embodiments, a composition for treating herpes simplex virus in a patient includes 300-450 mg of lithium carbonate every day for 3 to 7 days, or until blisters heal. In some embodiments, the lithium carbonate dose is 3 mg/kg up to about 15 mg/kg per day. In some embodiments, for patients greater than 50 kilograms (kg), the lithium carbonate dose is 300 to 450 mg/day. In some embodiments, for patients 10 to 50 kg, the lithium carbonate dose is 150 mg/day.

At this level of lithium, it is not necessary to monitor the levels of lithium carbonate in the patient's blood.

In some embodiments, the lithium carbonate is administered or co-administered to a patient along with an anti-viral. Non-limiting examples of anti-viral drugs, include valcyclovir and acyclovir. In some embodiments, an anti-viral drug is administered at a dose of about 300 mg.

In some embodiments, a composition includes lithium carbonate as described above in combination with an anti-viral drug. In some embodiments, a kit includes lithium carbonate and an anti-viral drug as described above.

In some embodiments, a method of treating post-herpetic neuralgia in a patient includes administering 300-450 mg of lithium carbonate every day for 3 to 7 days. The lithium carbonate dose is 3 mg/kg up to about 15 mg/kg per day. In some embodiments, for patients greater than 50 kilograms (kg), the lithium carbonate dose is 300 to 450 mg/day. In some embodiments, for patients 10 to 50 kg, the lithium carbonate dose is 150 mg/day. In some embodiments, the lithium carbonate is administered or co-administered to a patient along with an anti-viral.

In some embodiments, a method of reducing incidence of recurrence of herpes simplex virus 1 or 2 in a patient includes administering 300-450 milligrams (mg) of lithium carbonate every day for 3 to 7 days. The lithium carbonate dose is 3 mg/kg up to about 15 mg/kg per day. In some embodiments, for patients greater than 50 kilograms (kg), the lithium carbonate dose is 300 to 450 mg/day. In some embodiments, for patients 10 to 50 kg, the lithium carbonate dose is 150 mg/day. In some embodiments, the lithium carbonate is administered or co-administered to a patient along with an anti-viral.

Imbalance of Dopamine

In some embodiments of the present invention, a composition for treating an imbalance of dopamine in a subject having an imbalance of dopamine, without inducing post-synaptic sensitivity in the subject, includes an effective dose of haloperidol and an effective dose of amantadine. In some embodiments, an effective dose of haloperidol is from about 0.2 mg to about 10 mg per day. In some embodiments, an effective dose of amantadine is from about 10 mg to about 50 mg per day.

In some embodiments, for treating an imbalance of dopamine in a subject having an imbalance of dopamine, without inducing post-synaptic sensitivity in the subject, includes an effective dose of haloperidol, an effective dose of amantadine, and an effective dose of alpha-methyl-para-tyrosine, metyrosine (AMPT). For example, an effective dose of AMPT is from about 25 to 100 mg/day.

In some embodiments the subjects having an imbalance of dopamine suffer from tardive dyskinesia, tics, and/or Tourette's disorder. Examples of post-synaptic sensitivity include attention deficit hyperactivity disorder (ADHD), obsessive compulsive disorder (OCD), extreme anxiety, panic attacks, dysgraphia, anorexia, and/or tantrums (including exorcist tantrums).

In some embodiments, a kit for treating tardive dyskinesis, tics, and/o Tourette's disorder, includes an effective dose of haloperidol and an effective dose of amantadine. In some embodiments, the kit also includes an effective dose of AMPT.

In some embodiments, a method for treating a subject having an imbalance of dopamine, includes administering or co-administering an effective dose of haloperidol and amantadine. In some embodiments, the method also includes administering AMPT.

In some embodiments of the present invention, the method for treating a subject having an imbalance of dopamine with haloperidol and amantadine with or without AMPT as described above for at least one month. In some embodiments, after one month, the dose of haloperidol is decreased by 0.5 mg/day and administered for one month, followed after the second month, by a decrease in the dose of haloperidol by 0.5 mg/day and administered for one month. This titration of haloperidol is repeated until the haloperidol is no longer administered. Similarly, the dose of amantadine may be decreased in 0.5-5 mg/day increments on a monthly basis.

The action of haloperidol includes masking the symptoms by diminishing post-synaptic dopamine. The combination with amantadine prevents or reduces the incidence of post-synaptic sensitivity, examples of which are disclosed herein.

In some embodiments of the present invention, an imbalance of dopamine in a subject is treated with a composition including an effective dose of AMPT. In some embodiments, the effective dose of AMPT is from about 25 to about 100 mg/day. As disclosed, AMPT may be administered to treat dopamine imbalances including tardive dyskinesia, tics, Tourette's disorder, episodic vomiting disorder, pediatric acute onset neuropsychiatric syndrome (PANS), pediatric autoimmune neuropsychiatric disorders associated with streptococcal infections (PANDAS), and combinations thereof.

In some embodiments, a subject having PANS or PANDAS is treated with an effective dose of AMPT. An effective dose of AMPT includes about 25 to about 100 mg/day. In some embodiments, an effective dose of AMPT for treating PANS or PANDAS includes about 25 to about 50 mg/day AMPT.

In some embodiments, a composition for treating an imbalance of dopamine in a subject having an imbalance of dopamine, without inducing a histaminergic response, includes an effective does of amantadine. In some embodiments, the the imbalance of dopamine occurs in an indication of pediatric acute onset neuropsychiatric syndrome (PANS) or pediatric autoimmune neuropsychiatric disorders associated with streptococcal infections (PANDAS). In some embodiments, a composition of amantadine for treating PANS or PANDAS includes an effective dose of amantadine at about 10 mg to about 50 mg per day.

Obsessive Compulsive Disorder and Autism Spectrum Social Disorder

In some embodiments of the present invention, a composition for treating a subject having obsessive compulsive disorder (OCD) and/or autism spectrum social disorder without blocking dopamine, includes an effective dose of fluoxetine, and an effective dose of risperidone or aripiprazole.

Using a combination of fluoxetine and risperidone or aripiprazole, resolution of OCD is observed while stabilizing serotonin reactivity in the synapse. Additionally, by not using selective serotonin reuptake inhibitors (SSRIs), the side effects of SSRIs, such as weight gain and sleep disorders, are avoided.

Stabilization serotonin is critical to homeostasis. The many functions of serotonin include the regulation of mood, appetite, and sleep. Serotonin also has some cognitive functions, including memory and learning. Modulation of serotonin at synapses is thought to be a major action of several classes of pharmacological antidepressants.

In some embodiments, an effective dose of fluoxetine includes about 5 mg/day for three days/week up to about 20 mg/day.

In some embodiments, the effective dose of risperidone is 0.05 mg/day to 0.10 mg/day.

In some embodiments, the aripiprazole (Abilify®) is in tablet or syrup form, and the effective dose of the tablet form is 2.5 mg twice a week and the effective dose of the syrup form is 0.05 mg twice a week up to 0.5 mg/day

In some embodiments, a kit for treating OCD and/or autism spectrum disorder includes an effective dose of risperidone and an effective dose of aripiprazole in which aripiprazole is in pill or liquid (syrup) form as described above.

Schizophrenic Acute Psychosis and Autism Spectrum Social Disorder

In some embodiments of the present invention, a composition for treating a subject with schizophrenic acute psychosis and/or autism spectrum social disorder, without inducing psychosis, includes an effective dose of mecamylamine. In some embodiments, the effective dose of mecamylamine is 0.025 mg/kg to about 0.125 mg/kg twice a week.

In some cases, the effective dose of mecamylamine in schizophrenic acute psychosis patients and patients having autism spectrum social disorder results in improved verbal communication from 50 to 150%. In some embodiments, one dose may have an effect of improved verbal communication up to two months. In another example, one weekly protocol resulted in an effect of improved verbal communication for up to 4 months.

Dissociation Disorder

In some embodiments of the present invention, a composition for treating a subject with dissociation disorder, without inducing aggression or disinhibition, includes an effective dose of memantine. In some embodiments, an effective dose of memantine is 2.5 mg to 10.5 mg over 7 days.

Example Protocol for Memantine (Namenda®)

Day 1: Give Namenda ½ of a 5 mg tablet in the morning.

Day 2: If the patient has absolutely no positive change in her behavior in the form of genuine social interactions, give another ½ of a 5 mg tablet in the morning. Do not give the second dose if she even has slight improvement.

Day 3: No matter what happened, give no Namenda on Day 3.

Day 4: If there has been improvement, do not give the dose. If there has been no improvement, give ½ of a 5 mg tablet in the morning.

Day 5: If there has been no improvement since Day 1, give ½ of a 5 mg tablet in the morning. If you did not give Namenda on Day 4 and the patient is only slightly improved, give ½ of a 5 mg in the morning. If she has shown improvement after the Day 4 dose, do not give a dose on Day 5.

Day 6: No matter what happens, give no Namenda on Day 6.

Day 7: If there has been only minimal improvement after a total of 7.5 mg of Namenda, give ½ of a 5 mg Namenda. If there has been no improvement after a total of 10 mg of Namenda, give ½ of a 5 mg Namenda. Otherwise, give no Namenda on Day 7.

After the first week, if improvement is seen without signs of disinhibition, calculate the total dose for the first week and give that amount in divided doses throughout week 2.

Here is a sample chart for an example patient following the steps and a plan for week 2.

| | | | Degree or Detail | Side Effects or | Action for |
|---|---|---|---|---|---|
| Day | Dose given | Improvement | of Improvement | Disinhibition | next day |
| 1 | ½ of a 5 mg Namenda | None | | None | Give dose |
| 2 | ½ of a 5 mg Namenda | None | | None | No dose |
| 3 | None | Slight | Spoke normally | None | No dose |
| | | | to daughters for | | |
| | | | 1 hour. | | |
| 4 | None | Slight | Same as Day 3 | None | Give dose |
| 5 | ½ of a 5 mg Namenda | Good | Ate her meal | None | No dose |
| | | | without thinking | | |
| | | | about it. Instead | | |
| | | | of being forced | | |
| | | | to eat. | | |
| 6 | None | Continues Good | Same as Day 4 | None | No dose |
| 7 | None | Same | | None | See Below |

Protocol for 2nd week and beyond: During the first week, the above patient had 3 days of ½ of a 5 mg Namenda totaling 7.5 mg for the week. The second week's regime would be ½ of a 5 mg on Monday, Wednesday, and Friday mornings. As long as improvement is maintained at an acceptable level, the initial dosage can be maintained week by week. If the improvement wanes, slowly increasing the dose may help.

Side effects of Namenda: how to know if the dose is too high. The negative social effects of Namenda can be described as disinhibitory. This mean that the person's normal understanding of personal boundaries with others is lost—i.e., the patient loses their inhibition like someone who is intoxicated. They may be excessively aggressive both verbally and physically about incidental matters. They may become intrusive into other people's business. They may become flirtatious or aggressive. If a person has any of these behaviors it most likely means that the Namenda dose is getting too high.

Hypertension

A composition for treating a subject with hypertension without inducing fluid retention or fatigue in the subject, includes an effective dose of propranolol or timolol. The hypertension may be pre-hypertension, represented by a blood pressure of 130/90 millimeters of mercury (mm Hg) to 140/90 mm Hg. The hypertension may also be above 140/90 mm Hg.

By administering effective doses of propranolol or timolol at night, vasoconstriction caused by these drugs decreases intravascular volume while also blocking other symptoms common with excess beta reactivity such as tachycardia and gastroesophageal reflux disease (GERD).

In some embodiments, the subject having hypertension is taking other medications inlcuding first loop diuretics, calcium channel blockers (CCBs), thiazide diuretics. In some embodiments, a patient established with propranolol or timolol as above can be weaned from the other medications. If more than one medication is being taken, then only one medication is removed at a time, while the propanolol or timolol is maintained.

In some embodiments, the effective dose of propranolol is from 10 mg every other night to 40 mg every night.

In some embodiments, the effective dose of propranolol is from 10 mg every night to 20 mg every night.

In some embodiments, the effective dose of timolol is 5 mg every other night to 10 mg every night.

In some embodiments, a treatment for hypertension includes an effective does of propranolol or timolol as disclosed above at night and a vasoconstrictor medicament during the day. Examples of a vasoconstrictor medicament include an angiotensin-converting enzyme inhibitor (ACEI) or an angiotensin receptor blocker (ARB). Non-limiting examples of an ACEI include perindopril, captopril, enalapril, lisinopril, or ramipril, and non-limiting examples of an ARB include eprosartan, olmesartan, azilsartan medoxomil, telmisartan, losartan, valsartan, candesartan, or irbesartan.

Alpha Adrenergic Fatigue and Postural Hypotension

In some embodiments of the present invention, composition for treating a subject having alpha adrenergic fatigue or postural hypotension, without inducing hypotension in the subject, includes an effective dose of clonidine or an effective dose of venlafaxine.

In some embodiments, the effective dose of clonidine is 0.025 mg to 0.1 mg administered at night, and the effective dose of venlafaxine is 37.5 mg administered in the morning.

By treating alpha adrenergic fatigue or postural hypotension with clonidine or venlafaxine, the use of stimulants such as caffeine or nicotine may be avoided.

Sympathetic Dystrophy and Chronic Pain Syndrome

In some embodiments of the present invention, a composition for treating sympathetic dystrophy or chronic pain syndrome includes an effective dose of doxazosin and an effective dose of propranolol. This combination of doxazosin and propranolol at the effective doses is not addictive and does not cause flushing.

In addition, the combination of effective doses of doxazosin and propranolol maintains a partial block of the dilating effect of adrenaline on blood vessels. By inhibiting extreme dilation of blood vessels, the painful expansion of blood vessels is avoided, thereby inhibiting tissue swelling and pain.

For chronic pain syndromes, the combination of doxazosin and propranolol in effective doses is combined with non-narcotic pain treatments. Non-limiting examples of non-narcotic pain treatments include non-steroidal anti-inflammatory drugs (NSAIDS), nerve stabilizers, physical therapy, and combinations thereof.

In some embodiments, the effective dose of doxazosin is from 1 mg/day to 2 mg/day, and the effective dose of propranolol is 10 mg/day up to 10 mg three times a day.

In some embodiments, a kit for treating reflex sympathetic dystrophy or chronic pain syndrome, includes an effective dose of doxazosin and an effective dose of propranolol. The kit may also include additional non-narcotic treatments as described above.

Migraines

In some embodiments of the present invention, a composition for treating or reducing incidence of a migraine in a subject without inducing over dilation of blood vessels in the subject, includes an effective dose of propranolol, and an effective dose of doxazosin.

This combination of doxazosin and propranolol at the effective doses is not addictive and does not cause flushing.

In addition, the combination of effective doses of doxazosin and propranolol maintains a partial block of the dilating effect of adrenaline on blood vessels. By inhibiting extreme dilation of blood vessels, the painful expansion of blood vessels is avoided, thereby inhibiting tissue swelling and pain.

In some embodiments, the effective dose of propranolol is 10 mg/day up to 10 mg 4 times a day, and the effective dose of doxazosin is 1 mg/day to 2 mg/day. In some embodiments, the form of propranolol is not long acting.

In some embodiments, a composition for treating migraines includes an effective dose of clonidine alone or in combination with an effective dose of doxazosin. An effective dose of clonidine is 0.025 mg to 0.1 mg.

In some embodiments, a kit for treating or reducing the incidence of a migraine in a subject, without inducing over dilation of blood vessels in the subject, includes an effective dose of doxazosin and an effective dose of propranolol. The kit may also include an effective dose of clonidine and doxazosin.

### ADDITIONAL EXAMPLES

Changing the set point of a neurosynapse by changing agonist and antagonist by small amounts

Example: Experimentation of tardive dystonia with haloperidol and amantadine.

Demonstration of Techniques for Slow Change of Set Point

1. Four approaches to eliminate tardive movement and behavior disorders.

a) JOLT—Every other weekend lowered neuroleptic 10%, but on those weekends expected behavioral withdrawal symptoms.

b) CONTRA COUP Also lowered neuroleptic 10% and added 4 days of bromocriptine to stimulate pre-synaptic auto-receptors to lower dopamine output. These would normalize.

c) BOUNCE Lower the medication 1/10 of the original dose every month starting 2 days a week then 4 days a week then 6 days a week and then every day.

d) SMALL GAP Add agonist to regime after masking with neuroleptic then lower neuroleptic gradually

All had the same principle, slow changes but with protection from high activity at the time of change. Glucose metabolism is influenced by the same principle and a person with stable diabetes can train their blood sugar and insulin resistance to be lower.

Measured blood levels may not be as significant in treatment as the change in the blood levels

Maintaining low blood levels in bipolar and seizure disorder patients when symptoms are in remission can allow for a high dose of medication to be given when acute symptoms occur and thus strongly affect the acute symptom.

Similarly, slowly lowering the dose of the medicine to control episodes when patient is in remission in bipolar and seizure disorder patients when patients are in remission can allow for a high dose of medication to be given when acute symptoms occur and thus adequate treatment for the acute symptoms.

As an example, psychosis is most often thought to be a result of as too much dopamine. For example the L-dopa can cause hallucinations in Parkinsonism. Schizophrenics have active hallucinations somehow related to excess dopamine being available. However, psychosis can occur in depression and older people. Then it often is not controlled if treated with a dopamine blocker. In fact, it often gets worse.

Potential pathology of extreme change and compensates when effect will be pathological

Example: Treating vascular diseases such as migraine and reflex sympathetic dystrophy with both alpha and beta blockers to limit the vessel from extreme vasodilatation or constriction. These three descriptions help us understand that the body is not regulated by the blood level alone.

Some disorders are not due to a constant pathological condition, but on a cycle of physiological change during the diurnal cycle

Example: The use of SSRI antidepressant in certain patients during the last week of the menstrual cycle for pre-menstrual Syndrome.

Example: Treatment of hypertension with nighttime beta-blocker causing nighttime vasoconstriction, which eliminates nocturnal hypervolemia.

Introduction of Two Little Known Phenomena:

1. The overriding of the rate limiting of dopamine production by tyrosine hydroxylase when stimulated by NE, histamine, nicotine, and others. The enzyme is made more active.

2. The overriding of the rate limiting of dopamine production by tyrosine hydroxylase when stimulated by NE histamine, nicotine, and others explain the irregularity of dopamine oversensitivity/over-reactivity of increased DA causing episodic side effects such as tics, extrapyramidal symptoms and tardive dyskinesia.

This change in efficiency of dopamine manufacture allows the dopamine synapse to suddenly change its reactivity increasing the intensity of psychotic rage etc. It also gives increased movement disorder such as tics. These bursts of excess dopamine prevent the synapse from stabilizing because changes in emotional reactivity changed the dopamine level.

This excess dopamine actually stays in the cytoplasm of the cell and is not part of the granular storage of dopamine. Thus regular dopamine production.

Individuals who have nearly constant excess alpha-tone (constant stress) will often have hypervolemia and postural hypotension: this syndrome, while very common, is not a recognized disorder by most physicians. The understanding of this disorder helps validate the diurnal nature of hypertension.

Further understanding of hypovolemic states: The circulatory difficulties are weakness and dizziness on standing. Over time the excess effort needed for fighting these symptoms may put excessive pressure on the heart to beat more forcefully and may produce symptoms to the heart especially damage to the mitral valve. When treated with clonidine 0.1 mg as the typical dose, it allows for relaxation of mental tension and also dilatation of blood vessels expanding the volume of the vascular bed. (Clonidine is a vaso-dilator—it should not be thought of as an anti-hypertensive.)

Buffered Neuron Concept

The concept of the “buffered” neuro-chemical system (feedback mechanisms) is the property that maintains stability of brain function. Nerves do not function as individual/independent cells. They are surrounded by many other nerves and non-nerve filler cells (glia). A nerve's being stimulated is not a clean hit like a home run in baseball. Each time the nerve fires, it affects multiple associated cells. The process is more like capturing the king in a chess game where every change creates a potential for increased or decreased likelihood of the king's capture. This situation has caused the disciplines of psychiatry and neurology to have many unanswered questions.

The glutamate receptor is one of these quizzically reacting systems. Few medications have been successful in treating patients where glutamate is involved. There are few disorders we identify as being affected by glutamate despite the large role-played by glutamate in the brain chemistry. One medication that has been produced is Memantine, which is supposed to help memory in patients with Alzheimer's disease. However its effect in many people is to increase their socialization, thus making them seem more mentally bright.

A psychological phenomenon potentially related to glutamate is dissociation. Dissociation has not been widely recognized by the medical community; though psychologists and therapists regularly help patients identify their behaviors with this term. Most hallucinogenic drugs, whether market or recreational, affect glutamate. Hallucinations are one form of dissociation. The dissociative state can last for years and may be a product of the highly buffered nature of the glutamate receptor.

Scientific reasons derived from experimental evidence, which are the basis for the new methodologies:

1. Actual case patient with seizures, anti-conversant toxicity or “acting out”.

2. Discovery of beta rage and alpha rage distinction.

3. A hyperactive individual with low blood pressure and pulse.

4. Determining to cure tardive dyskinesia using slow withdrawal methods.

5. Method of preventing withdrawal rebound phenomenon by preventing high levels on either side of the synapse.

6. Use of “Trojan horse” doses with a complex synapse by using minute dose of some medication, which causes change in the cell.

For example one of the stimulating systems in the body is the glutamate receptor mechanism. Some of the biggest challenges in neuropsychiatric pharmacology is breaking through the defenses of these “buffered” systems like NMDA. The “Trojan horses” can enter into the receptor mechanism and change it without disturbing the situation. This explains why certain diseases that are not well treated, such as autism, have responded to any number of treatments but none of them consistently. If the balance between more autistic and less autistic behavior is very much leaning toward improved socialization, then perhaps any “Trojan horse” that can slip past the “fortress” of the glutamate receptors that are refusing to participate may add enough positive to affect socialization and the child has a miraculous response to the minute dose of medicine.

Examples: low dose Abilify and mecamylamine have been successful in treating patients where involvement with glutamate is suspected. Indeed we have few disorders that are known to be caused by glutamate despite the large role it plays in brain function. One medication that has been used for several years is Mematine, which is supposed to help memory in patients with Alzheimer's. However, its effect on many people is increasing their socialization and thus making them seem more mentally brightened. Another example is the destructive flashbacks seen when individuals have taken PCP. These flashbacks may occur for years long after the PCP should have been eliminated from the body.

The NMDA receptor is one of the glutamate receptors. It is quite complex. First of all in order for it to fire it must be stimulated by d-serine which is made from the amino acid l-serine. L-serine is found in abundance in the body. However the NMDA receptor will not recognize the l-serine around it because it requires the mirror image chemical d-serine meaning rotating to the right. In order to do that it must be converted from its present left-handed to right-handed. There are a number of filler cells (called glia), which have the property of turning the left-handed l-serine into the mirror image right-handed. Nowhere else in the body is there an enzyme to convert left-handed to right-handed, but that special d-serine key doesn't begin to expose all the chemical changes that can occur.

Other receptors, which have complicated feedback mechanisms, are the opioid receptor or opium based pain medication, the so-called benzodiazepine receptor that is affected by drugs like Valium.

When these complex “buffering” systems carry out their functions it prevents a significant change from occurring. Rather than having a “jumpy” mechanism that will respond, these receptors will fail to change because they are protected by the other receptors.

Interestingly, the nerve cells with the most complex “buffering” mechanism of their receptors are the ones stimulated by the drugs, which are most addictive. (Dopamine receptor and alpha-l receptor—cocaine. Opioid receptor—heroin.) In a sense we might say that a patient with tardive dyskinesia has developed a haloperidol addiction because if that body does not take the haloperidol the tardive movements will occur again.

The “Trojan Horse” concept (overcoming the neuronal “buffering” system)

The Trojan Horse complex appears to explain phenomena that are seen frequently and have remained unexplained.

Some of the biggest challenges in neuropsychiatric pharmacology is breaking through the defenses of these “buffered” systems like NMDA.

The “Trojan horses” can enter into the receptor mechanism and change it without disturbing the protective systems. This explains why some individuals with certain diseases currently are not well treated, such as autism. Some individuals have responded to any number of treatments but none of them consistently. If the balance between more autistic and less autistic behavior is very much leaning toward improved socialization, then perhaps any “Trojan horse” that can slip past the “fortress” of the glutamate receptors, that are refusing to participate, may add enough positive to affect socialization and the child has a miraculous response to the minute dose of medicine.

Association Model of Brain Function

This section presents a view of how the brain works—at least how it functions chemically. The complex nerve pathways of the brain are an important part of the brain's ability, but the actual distinction between functional and dysfunctional brains always involves a chemical reaction.

Association model of brain function: The brain's function depends on continuous monitoring by the “internet” of brain cells and being aware of the variation in the signals being received. The “centers” communicate with each other using variable amplification or suppression of the signals each receives. The brain thus has a holograph like function—every part influenced by every other part producing a varying momentary image through this continuous association.

Dissociation: dissociation is a normal protective mechanism to eliminate “toxic” situations, which would disturb the smooth function of the brain. Dissociation is fairly common with intense pain, illness, or social estrangement. It is peculiar in being a clinically recognizable dysfunction, but seemingly does not have a chemical cause or treatment. Many psychiatrists even doubt the existence of dissociative state.

Dissociation and glutamate: There are some indicators that dissociation is related to variations of glutamate activity. Ketamine is an anesthetic commonly used for children. It causes individuals to lose coherent ideas. PCP is also a glutamate acting drug. It is associated with “bad trips” which can recur for years. Episodes of dissociation can have a peculiar tendency to last for a long time—even years. It may come and go away one time or repeat during a particular recurring event. It is normally considered a product of emotional conflict; yet often goes away in relation to a particular event. This ability to maintain long term “disorders” or instantly dismiss them is not typical neurological function. It could possibly be a property of the glutamate neurons. The cures for dissociative episodes are often psychologically meaningful events or peculiar pharmacological products. In other words the conversion reaction remains stable until “jolted.” The “jolt” is idiosyncratic to the patient.

Autism and Dissociation, Autism and the “Socialization Factor”

The realm of autism may not be as mysterious as we perceive it to be. If Dr. Margaret Mahler is right about the psychological development of the child, then all brains start off with an autistic like separation of emotional reaction to the outside world. Most children are quickly attracted to the stimulation of the outside world because it is more pleasurable than autistic isolation. Any irregular physical, environmental, or psychological difficulty may prevent the developing child from moving into socialization. Thus, autism develops when the brain associations remain focused on internal stimuli instead of moving to the external, social world. If the child remains in his autistic state, he will continue to withdraw from social interaction and become highly estranged from the world. This estrangement prevents social growth leading to increased emotional isolation and the spectrum of autistic symptoms.

Treating autism: All autistic children seem to have some capacity for more socialization than they demonstrate. Seemingly, the autistic control of the brain prevents it from accepting emotional intimacy and the subsequent socialization, but occasionally an individual will spontaneously act with normal social intention. For example the autistic patient will say to an injured sibling: “Don't cry, honey, it will feel better.”

Autism and Socialization

There are over one hundred statements by various physicians of some treatment that improves the socialization of autistic individuals: there are over a thousand by other individuals invested in autistic care. None of these treatments improves all individuals with autism. These treatments have one thing in common—when there is an effect it is immediate and results in a radical behavior change. This is best explained by the understanding that the social personality is repressed and the treatment involved in change triggers that patient's idiosyncratic need, which allows for increased socialization. This socialization is sometimes permanently improved. Most of the time, however, the change is brief.

Discussion of Clinical Observations:

1. Analyzing unusual presentation of symptoms with known scientific understandings.

Case Number 1: The seizure patient with symptoms of over medication, under medication, and “acting out.”

2. The scientific basis for the belief that alpha blockade may be necessary to stop some rage behavior.

Case number 2: A seizure patient with rage behavior had less frequent rages on the beta blocker propranolol, but when he had a rage it was wilder and more psychotic than before beta blocker treatment. The patient had been on a large dose thioridazine (400 mg/day). Because the propranolol and the thioridazine block the same hepatic enzyme, thioridazine was discontinued and patient was placed on haloperidol, which does not have the strong alpha adrenaline blocking effect that thioridazine has.

Scientific literature reveals two types of rage represented by two separate columns of cells in the brain stem. One drives fear and involves beta-receptors. The other drives aggression and involves alpha-receptors. Addition of the alpha blocking medication doxazosin stopped the severe psychotic symptoms.

3. Alpha-adrenergic rage occurs by changes in the blood vessels of the brain which then changes brain cell function.

Scientific literature demonstrates that high levels of norepinephrine in meningeal arteries cause break down in the blood-brain barrier.

Clinical study: A nonselective beta-blocker may allow for a patient's alpha rage to continue by causing vasoconstriction. A highly beta-l selective beta-blocker will not produce this untoward effect.

4. Use of agonist together with antagonist to minimize strength of response.

5. Use of medications according to diurnal cycle.

6. Complex “buffered” synapses can be highly resistant to change as in dissociative phenomenon and flashbacks.

7. Similar symptoms may be caused by a low level of neurotransmitter or by a high level of neurotransmitter. It is the imbalance between pre-synaptic and post-synaptic neurons that results in the symptom. Examples: Tourette's disorder, tardive dystonia vs. EPS dystonia, “Burned out” Schizophrenics

Additional Diseases/Disorders/Conditions that can be Treated with the Disclosed Methods.

Post Menopausal Syndrome presents a delta so extreme that the points are not consistent enough to regulate with current medications. By applying the Delta Treatment Protocol, the varying extremes of hormonal impulses that produce the imbalance can be re-regulated by applying both suppression and stimulation.

Diabetes: People who become resistant to insulin are currently being treated by providing more insulin. The outcome is further insulin resistance. The delta of the insulin/glucose balance becomes more extreme, refractory to treatment. Applying delta treatment, one can eventually train the levels back to a manageable and consistent basis, giving the pancreas renewed regulation.

Nicotine Addiction: Highly addicted people can be treated by applying high dose AMPT. Nicotine enhances dopamine receptor sensitivity, stimulating higher production and generating experience of euphoria. The delta slope is thereby made steeper. Applying delta theory as well as AMPT as a Trojan Horse, one can wean a patient off of nicotine and create a new set-point, eventually titrating off of the medication.

Currently, infection triggered mental illness and autoimmune diseases such as tic disorders, tardive dyskinesia, and Tourette's can be treated by reducing reactivity to immunological threats. This can be viewed as a delta between the stimulation of the body's immune reaction and histaminergic response and the suppression of that response with medication. The autoimmune response is already heightened to stimuli and that sensitivity is triggered by pathogens that are not necessarily detectable with current common testing practices.

Medication that currently exist and used for treating vascular based disorders are contraindicated in patients that suffer from asthma and other pulmonary diseases due to the broncho-constriction or spasm that is a reaction to the medication. A new pharmaceutical agent that can predictably not trigger bronchial reactions can treat these patients successfully.

Currently severe cystic acne is treated by giving antibiotics, which does not kill the bacteria, it only prevents it from multiplying. The problem most identified with acne is the problem of permanent scarring, which is a result of normal hair follicles becoming impacted with sebum (blackhead) as well as actively infected follicles (whitehead). By giving a patient antibiotics 24 hrs a day, the set-point is not changed between the bacteria and the antibiotic, allowing the bacteria to multiply more rapidly than the antibiotic can kill, allowing the further growth abscesses. The best result obtained to date reduces the number of abscesses by slightly better than 50%. Acne is actually a cyclical/rhythmic disorder, where outbreaks will wax and wane, at a low point antibiotic can be stopped. When an outbreak wanes, the antibiotic can be doubled to further suppress the bacteria. This cuts the colony reproduction from one half to one eighth. Utilizing a topical hydrocortisone patch after cleaning the site with warm soapy water, the re-colonization of bacteria is halted, the inflammation is lowered and the cycles of infection/inflammation are fewer and of less severity, eventually resetting the set point of the bacterial infection.

Obesity and Inflammatory Bowel Disease: Relates to the glycemic curve. We eat high carbohydrate foods as we crave the energy provided. A rebound effect is produced leading to the eating of more high carbohydrates. If the digestive process of carbohydrate metabolism is delayed by applying digestive enzymes and slow release amylase inhibitors, the glycemic curve is kept modulated, stopping a steeper delta from dominating digestion. This also assists in pancreatic regulation that in turn modulates insulin resistance. This entire treatment, whether for obesity or diabetics intolerant to medication, can be packaged with a smart-phone or tablet type of application that would contain algorithms that would indicate what medication to take at a particular time to assist in controlling this function thereby resetting the set-point. This application could also apply to Inflammatory Bowel Disease and other inflammatory autoimmune diseases, modulating and resetting the balance of inflammatory response. In many of these disorders, a Trojan Horse treatment can be applied to wean the patient of medication once a dyregulated set-point has been resolved.

Shingles can managed in a similar manner with Lithium. Giving low dose Lithium for 4 days within 36 hours of initial outbreak and viral replication can be stopped quickly. Current practice is only antiviral therapy along with pain management and outbreaks can continue for weeks.

Condiloma/HPV/Genital Warts (also applying to common warts): Current treatment is burning/freezing or etching with salicylic acid whenever there is an outbreak. Applying the Delta Theory, the virus can be treated externally and internally simultaneously, using an agent like superglue over and around the wart surface causing the cells to die and therefore rendering the virus incapable of being transported by the blood supply and replicating. Simultaneously treating with low dose lithium addresses the internal viral load. This process should be repeated in one month.

Autism, disorders of disassociation and lack of socialization: Can also be treated with Delta Theory, by resetting the neural pathways with low dose Abilify. The low dosage is key to affecting neural pathways by awakening socialization. Similarly, looking at memory disorders with dysregulated set points of neural pathways, Delta Theory can be applied using existing pharmaceutical agents in new and different ways. An experiment with a patient that suffered traumatic brain injury and lost the ability to communicate for ten years was treated with this method of resetting the neural pathways and regained communicative abilities.

Mental Illness, Bipolarity and Mood Disorders, Schizophrenia, OCD, Anxiety Disorders, Panic Attacks, Psychosis: All are based in dopamine dysregulation. These can be treated and tightly controlled with Delta Theory, applying a variety of medications in low dose usage and Rational Poly-Pharmacy, and can be titrated off medication once re-setting of the set-point has been achieved. The application of agonist/antagonist medications in these disorders can effectively reset a dyregulated set-point.

Common practice today is administering medications that often compete with each other because they are applied linearly. This approach is absent of Delta Theory and demonstrates that this is not yet understood or applied. The concept of Rational Poly-pharmacy uses lower dose medications to address a given disorder with a complete envelope. Many medication regimens fail because they do not recognize or treat this envelope in its entirety. Instead, common practice can create an exacerbated delta by relieving symptoms short term but creating long term problems.

Unique components of Delta Theory and Rational Poly-pharmacy is that they examine the physical body and its relationship to dysregulation of brain chemistry. Here is a new and different way to examine and understand certain neuropsychiatric behaviors, whereby adrenaline driven alpha or beta dysfunction and rages have been previously diagnosed as neuropsychiatric in nature. These instances can be described as adrenergic dysfunction as opposed to dopaminergic. This leads to an entirely different method of diagnosis as well as treatment, where the application of Delta Theory is particularly effective.

The disclosures of each patent, patent application, and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating a subject having a disorder or indication, comprising:
administering to the subject a minimal dose of a medicament necessary to alleviate the disorder based on action of the medicament.

2. A composition for treating herpes simplex virus, comprising:
an effective dose of lithium carbonate that dose not induce effects selected from the group consisting of tremor of the fingers, polyuria, sodium depletion, and central nervous system dysfunction.

3. The composition of claim 2, wherein the effective dose of lithium carbonate is from about 3 mg/kg up to about 15 mg/kg per day.

4. The composition of any one of claims 2-3 further comprising an anti-viral medicament.

5. The composition of claim 4, wherein the anti-viral medicament is selected from the group consisting of valcyclovir, acyclovir, and combinations thereof.

6. A kit for treating herpes simplex virus, comprising the composition of claim 4.

7. A method of treating herpes simplex virus in a subject infected with herpes simplex virus, the method comprising:
administering lithium carbonate to the subject.

8. The method of claim 7, further comprising:
administering an anti-viral drug.

9. The method of any one of claims 7-8, wherein the lithium carbonate is administered in a dose of about 3 mg/kg up to about 15 mg/kg per day.

10. A method of treating post-herpetic neuralgia in a subject having been infected by herpes virus, comprising:
administering the composition of any of claims 2-5.

11. A method of inhibiting recurrence of herpes virus symptoms in a subject having been infected by herpes virus, comprising
administering the composition of any of claims 2-5.

12. A composition for treating an imbalance of dopamine in a subject having an imbalance of dopamine, without inducing post-synaptic sensitivity in the subject, comprising:
an effective dose of haloperidol; and
an effective dose of amantadine.

13. The composition of claim 12, further comprising
an effective dose of alpha-methyl-para-tyrosine, metyrosine (AMPT)

14. The composition of any of claim 12 or 13, wherein the effective dose of haloperidol is from about 0.2 mg to about 10 mg per day, the effective dose of amantadine is from about 10 mg to about 50 mg per day, and the effective dose of AMPT is from about 25 to 100 mg/day.

15. The composition of any of claims 12-14, wherein the imbalance of dopamine occurs in an indication selected from the group consisting of tardive dyskinesia, tics, and Tourette's disorder.

16. A kit for treating tardive dyskinesia, tics, and/o Tourette's disorder, comprising:
the composition of any of claims 12-14.

17. A method of treating a subject having tardive dyskinesia, tics, and/or Tourette's disorder, the method comprising:
administering the composition of any one of claims 12-14.

18. The method of claim 17, further comprising:
decreasing the dose of haloperidol every month.

19. The method of claim 18, further comprising:
decreasing the dose of amantadine every month.

20. A composition for treating an imbalance of dopamine in a subject having an imbalance of dopamine, without inducing post-synaptic sensitivity in the subject, comprising:
an effective dose of alpha-methyl-para-tyrosine, metyrosine (AMPT).

21. The composition of claim 20, wherein the effective dose is from about 25 to about 100 mg per day.

22. The composition of any of claim 20 or 21, wherein the imbalance of dopamine occurs in an indication selected from the group consisting of tardive dyskinesia, tics, Tourette's disorder, episodic vomiting disorder, pediatric acute onset neuropsychiatric syndrome (PANS), pediatric autoimmune neuropsychiatric disorders associated with streptococcal infections (PANDAS), and combinations thereof.

23. A method of treating a subject having at least one indication selected from tardive dyskinesia, tics, Tourette's disorder, episodic vomiting disorder, pediatric acute onset neuropsychiatric syndrome (PANS), and pediatric autoimmune neuropsychiatric disorders associated with streptococcal infections (PANDAS), the method comprising:
administering the composition of any one of claims 20-21.

24. The method of claim 23, wherein the indication is PANS or PANDAS and the effective dose is from about 25 to about 50 mg/day.

25. A composition for treating an imbalance of dopamine in a subject having an imbalance of dopamine, without inducing a histaminergic response, comprising:
an effective dose of amantadine.

26. The composition of claim 25, wherein the imbalance of dopamine occurs in an indication of pediatric acute onset neuropsychiatric syndrome (PANS) or pediatric autoimmune neuropsychiatric disorders associated with streptococcal infections (PANDAS).

27. The composition of any of claim 25 or 26, wherein the effective dose of amantadine is about 10 mg to about 50 mg per day.

28. A method of treating a subject having PANS or PANDAS, comprising:
administering the composition of any of claims 25-27.

29. A composition for treating a subject having obsessive compulsive disorder (OCD) and/or autism spectrum social disorder without blocking dopamine, comprising:
an effective dose of fluoxetine; and
an effective dose of risperidone or aripiprazole.

30. The composition of claim 29, wherein the effective dose of fluoxetine is about 5 mg/day for three days/week up to about 20 mg/day.

31. The composition of claim 29, where the effective dose of risperidone is 0.05 mg/day to 0.10 mg/day.

32. The composition of claim 29, wherein the aripiprazole is in tablet or syrup form, and the effective dose of the tablet form is 2.5 mg twice a week and the effective dose of the syrup form is 0.05 mg twice a week up to 0.5 mg/day

33. A kit for treating OCD and/or autism spectrum social disorder, comprising:
the composition of any of claims 29-32.

34. A composition for treating a subject with schizophrenic acute psychosis and/or autism spectrum social disorder, without inducing psychosis, comprising:
an effective dose of mecamylamine.

35. The composition of claim 34, wherein the effective dose is 0.025 mg/kg to about 0.125 mg/kg twice a week.

36. A method of treating a subject with schizophrenic acute psychosis and/or autism spectrum social disorder, without inducing psychosis, comprising:
administering a composition of any one of claims 34-35.

37. A composition for treating a subject with dissociation disorder, without inducing aggression or disinhibition, comprising:
an effective dose of memantine.

38. The composition of claim 37, wherein the effective dose is 2.5 mg to 10.5 mg over 7 days.

39. A method of treating a subject with dissociation disorder without inducing aggression or disinhibition, comprising:
administering the composition of any of claims 37-38.

40. The method of claim 39, wherein the administering occurs over 7 days and comprises:
administering 2.5 mg of memantine on Day 1;
optionally administering 2.5 mg of memantine on Day 2;
withholding memantine on Day 3;
optionally administering 2.5 mg of memantine on Day 4;
optionally administering 2.5 mg of memantine on Day 5;
withholding memantine on Day 6; and
optionally administering 2.5 mg of memantine on Day 7.

41. A composition for treating a subject with hypertension without inducing fluid retention or fatigue in the subject, comprising:
an effective dose of propranolol or timolol.

42. The composition of claim 41, wherein the effective dose of propranolol is from 10 mg every other night to 40 mg every night.

43. The composition of claim 42, wherein the effective dose of propranolol is from 10 mg every night to 20 mg every night.

44. The composition of claim 41, wherein the effective dose of timolol is 5 mg every other night to 10 mg every night.

45. The composition of claim 41, wherein the hypertension in the subject is at least 130/90.

46. A method of treating a subject having hypertension, without inducing fluid retention or fatigue in the subject, comprising:
administering the composition of any of claims 41-45.

47. The method of claim 46, further comprising:
administering a vasoconstrictor medicament during the day.

48. The method of claim 47, wherein the vasoconstrictor medicament is selected from an angiotensin-converting enzyme inhibitor (ACEI) or an angiotensin receptor blocker (ARB).

49. The method of claim 48, wherein
the ACEI is selected from the group consisting of perindopril, captopril, enalapril, lisinopril, and ramipril; and
the ARB is selected from the group consisting of eprosartan, olmesartan, azilsartan medoxomil, telmisartan, losartan, valsartan, candesartan, and irbesartan.

50. A composition for treating a subject having alpha adrenergic fatigue or postural hypotension, without inducing hypotension in the subject, comprising:
an effective dose of clonidine or an effective dose of venlafaxine.

51. The composition of claim 50, wherein the effective dose of clonidine is 0.025 mg to 0.1 mg at night, and the effective dose of venlafaxine is 37.5 mg in the morning.

52. A method of treating a subject having alpha adrenergic fatigue or postural hypotension, without inducing hypotension in the subject, the method comprising:
administering the composition of any of claims 50-51.

53. A composition for treating reflex sympathetic dystrophy or chronic pain syndrome, comprising:
an effective dose of doxazosin; and
an effective dose of propranolol.

54. The composition of claim 53, wherein the effective dose of doxazosin is from 1 mg/day to 2 mg/day, and the effective dose of propranolol is 10 mg/day up to 10 mg three times a day.

55. A kit for treating reflex sympathetic dystrophy or chronic pain syndrome, comprising:
the composition of any of claims 53-54.

56. A composition for treating or reducing incidence of a migraine in a subject without inducing over dilation of blood vessels in the subject, comprising:
an effective dose of propranolol; and
an effective dose of doxazosin.

57. The composition of claim 56, wherein the effective dose of propranolol is 10 mg/day up to 10 mg 4 times a day, and the effective dose of doxazosin is 1 mg/day to 2 mg/day.

58. A kit for treating or reducing the incidence of a migraine in a subject, without inducing over dilation of blood vessels in the subject, comprising:
the composition of any of claims 56-57.

59. A method of treating a subject having a disorder or indication, comprising:
administering to the subject a minimal dose of a medicament necessary to alleviate the disorder based on action of the medicament.

60. A method of treating a subject having a disease, disorder, and/or condition comprising:
co-administering to said subject two or more agents, wherein the agents have opposing mechanisms of action.

61. The method of claim 60, wherein the agents comprising at least one agonist and at least one antagonist, wherein the agonist and antagonist regulate the same target molecule.

62. The method of any one of claim 60 or 61, wherein the target molecule comprises a cellular receptor or an ion channel.

63. The method of any one of claims 60-62, wherein at least one agent treats hypersensitivity and at least one agent that treats hyposensitivity.

64. The method of any one of claims 60-63, wherein the two or more agents are administered simultaneously.

65. The method of any one of claims 60-64, wherein the two or more agents are administered according to the diurnal cycle.
